(19) United States
(12) Plant Patent Application Publication
(10) Pub. No.: US 2012/0246774 P1
(43) Pub. Date: Sep. 27, 2012

(54) Pot Carnation Plant Named 'KLEDP10113'

(50) ;

(75) Inventors: Nils Klemm, Stuttgart (DE); Martin Glawe, Stuttgart (DE)

(73) Assignee: KLEMM+SOHN GMBH & CO. KG, Stuttgart (DE)

(21) Appl. No.: 13/065,498

(22) Filed: Mar. 23, 2011

(51) Int. Cl. A01H 5/00

(52) U.S. Cl. PLT/276

(57) ABSTRACT

A new variety of Carnation named ‘KLEDP10113’ particularly characterized by its light pink flowers; very early flowering; and good growth and performance, is disclosed.

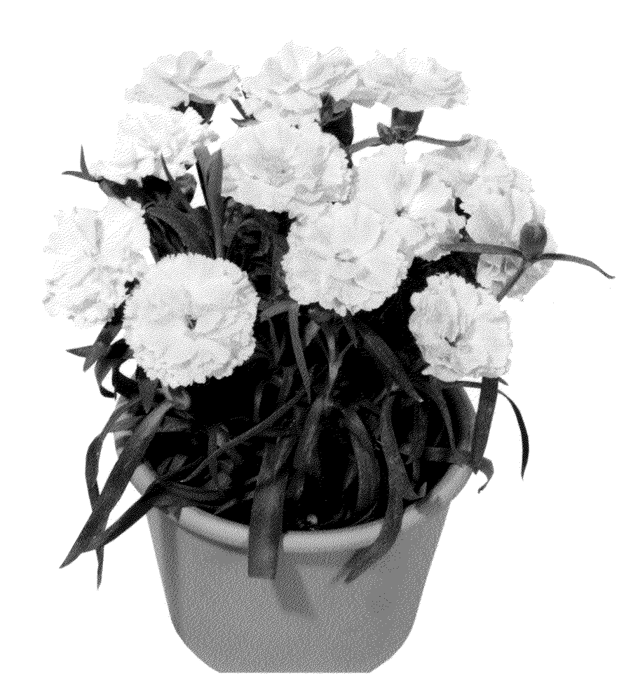

### GENUS AND SPECIES

Dianthus caryophyllus

### VARIETY DENOMINATION

‘KLEDP10113’

### BACKGROUND OF THE NEW PLANT

The present invention comprises a new and distinct variety of Carnation, botanically known as Dianthus caryophyllus, and hereinafter referred to by the variety name ‘KLEDP10113’. The new variety is the result of a cross conducted in June 2006 in Stuttgart, Germany between the proprietary female parent, ‘DCP 06 134’ (unpatented) and the proprietary male parent, ‘DCP 05 007’ (unpatented). A single plant selection was subsequently chosen for further evaluation and for asexual propagation.

The new variety was first propagated via vegetative cuttings in June 2007 in Stuttgart, Germany and has been asexually reproduced repeatedly by vegetative cuttings in Stuttgart, Germany for approximately six to seven generations. ‘KLEDP10113’ has been found to retain its distinctive characteristics through successive asexual propagations via vegetative cuttings.

Plant Breeder's Rights for this variety have not been applied for. ‘KLEDP10113’ has not been made publicly available or sold more than one year prior to the filing date of this application.

### SUMMARY OF THE INVENTION

The following are the most outstanding and distinguishing characteristics of this new variety when grown under normal horticultural practices in a glass greenhouse in Stuttgart, Germany.

1. Light pink flowers;

2. Very early flowering; and

3. Good growth and performance.

### DESCRIPTION OF THE PHOTOGRAPH

This new Carnation plant is illustrated by the accompanying photograph which shows a close-up of a mature flower, flower buds, and foliage. The colors shown are as true as can be reasonably obtained by conventional photographic procedures. The photograph was taken in April 2010 of a 5-month old plant grown from rooted cuttings in a 10 centimeter pot in a glass greenhouse in Stuttgart, Germany under normal horticultural practices.

### DESCRIPTION OF THE NEW VARIETY

The following detailed descriptions set forth the distinctive characteristics of ‘KLEDP10113’. The data which define these characteristics were collected from asexual reproductions carried out in Stuttgart, Germany. The plant history was taken in Spring 2010 on 5-month old plants grown in 10 centimeter pots with a minimum pinch date of 45 weeks. The plants were grown in a greenhouse covered with glass. The color readings were determined under natural light. Color references are to the RHS Colour Chart of The Royal Horticultural Society of London (R.H.S.), Fifth Edition (2007).

### DETAILED BOTANICAL DESCRIPTION OF THE NEW PLANT

Classification:
   Family.—Caryophyllaceae.
   Genus and species.—Dianthus caryophyllus.
   Common name.—Carnation.
Parentage:
   Female parent.—The proprietary Carnation ‘DCP 06 134’ (unpatented).
   Male parent.—The proprietary Carnation ‘DCP 05 007’ (unpatented).
Plant:
   Form.—Potted plant.
   Habit.—Upright.
   Height (from top of soil).—12.0 cm to 14.0 cm.
   Width (spread).—12.0 cm.
   Life cycle.—Perennial partially.
   Flowering habit.—Continuous.
   Time to produce a finished flowering plant.—5 months.
   Propagation type.—Vegetative cuttings.
   Time to initiate roots.—18 days.
   Root description.—White, fine.
   Temperature tolerances.—Normal.
Branches:
   Quantity per plant.—5 to 6.
   Color.—RHS 137A.
   Length.—4.0 cm to 8.0 cm.
   Diameter.—0.5 cm.
   Internode length.—0.5 cm to 1.0 cm.
   Texture.—Smooth.
Leaves:
   Arrangement.—Opposite.
   Shape.—Lanceolate.
   Apex.—Acute.
   Base.—Obtuse.
   Margin.—Entire.
   Color.—Immature leaf (both upper and lower surfaces): RHS 138A Mature leaf (both upper and lower surfaces): RHS 137A.
   Size.—Length: 4.0 cm to 6.0 cm Width: 0.4 cm to 0.6 cm.
   Texture.—Smooth.
   Venation color.—Upper surface: RHS 137A Lower surface: RHS 137C.
   Petioles.—Absent.
Flowers:
   Type.—Small sprays.
   Flowering season.—Spring to Fall.
   Size.—Width: 4.5 cm Depth: 1.5 cm.
   Shape.—Round.
   Fragrance.—Present.
   Number of flowers per plant.—4 to 7.
   Calyx.—Form: Cylinder.vase Length: 1.8 cm Width: 0.9 cm.
Flower buds:
   Color.—RHS 137B.
   Shape.—Elliptic.
   Length.—1.4 cm.
   Diameter.—0.7 cm.
Petals:
   Arrangement.—Arranged in several whorls.
   Quantity (per flower).—Greater than 18 to 25.
   Shape.—Spoon-shaped.
   Apex.—Obtuse.
   Base.—Acute.
   Size.—Length: 2.2 cm Width: 1.5 cm.
   Color.—When opening: Upper surface: RHS 55C with RHS 55A colored ring Lower surface: RHS 55D When fully opened: Upper surface: RHS 56A with RHS 55A colored ring Lower surface: RHS 56C.
   Texture (both upper and lower surfaces).—Smooth.
Sepals:
   Arrangement.—Fused.
   Quantity (per flower).—4.
   Shape.—Elliptic.
   Apex.—Acute.
   Base.—Obtuse.
   Margin.—Smooth.
   Size.—Length: 0.6 cm Width: 0.6 cm.
   Color.—Upper surface: RHS N138B Lower surface: RHS 138C.
   Texture (both upper and lower surfaces).—Smooth.
Peduncle:
   Length.—12.0 cm to 14.0 cm.
   Diameter.—0.2 cm to 0.3 cm.
   Texture.—Smooth.
   Color.—RHS 137B.
Reproductive organs:
   Pistils.—Quantity: 1 Stigma color: RHS 49D Ovary color: RHS 138C.
   Stamens.—Quantity: Moderate Filament: Length: 0.4 cm to 0.6 cm Color: RHS 49D Anther color: RHS 56C Pollen: Amount: Moderate Color: RHS 56D.
Fruit and seed set: None observed.
Comparision with parental and commercial varieties: ‘KLEDP10113’ differs from the female parent, the proprietary Carnation ‘DCP 06 134’ (unpatented), in that ‘KLEDP10113’ has a medium growth habit and light pink flowers, while ‘DCP 06 134’ has a compact growth habit and purple flowers. ‘KLEDP10113’ differs from the male parent, the proprietary Carnation ‘DCP 05 007’ (unpatented), in that ‘KLEDP10113’ has a medium growth habit and light pink flowers, while ‘DCP 05 007’ has a very strong growth habit and white flowers. ‘KLEDP 10113’ differs from the commercial comparison variety Carnation Plant Named ‘CFPC Pink Tess’ (U.S. Plant Pat. No. 21,364), in that ‘KLEDP10113’ has light pink flowers with a darker pink ring (RHS 55C and RHS 55A ring), an upright growth habit, and spoon-shaped flower petals, while ‘CFPC Pink Tess’ has purple pink flowers (RHS 62C), a semi upright growth habit, and fan-shaped flower petals.

We claim:

1. A new and distinct variety of Carnation plant named ‘KLEDP10113’ as shown and described herein.
